(19) United States
(12) Patent Application Publication
(10) Pub. No.: US 2026/0267930 A1
(43) Pub. Date: Sep. 10, 2026

(54) SEARCH PLATFORM FOR PROVIDERS FOR MEDICAL INJECTABLES

(71) Applicant: Jillian SARCONA, Locust Valley, NY (US)

(72) Inventor: Jillian SARCONA, Locust Valley, NY (US)

(21) Appl. No.: 19/071,261

(22) Filed: Mar. 5, 2025

(51) Int. Cl. G06F 16/9535; G06F 16/2457; G06F 16/9538; G16H 20/17; G16H 40/20

(52) U.S. Cl. CPC G06F 16/9535; G06F 16/24578; G06F 16/9538; G16H 20/17; G16H 40/20

(57) ABSTRACT

A system for a search platform for providers for medical injectables, includes a processor and a memory including instructions which, when executed by the processor, cause the system to: receive a first patient input indicating two or more of: a treatment type, an area to be treated, a treatment goal, or a geolocation; determine a list of providers based on the at least two of: the treatment type, the area to be treated, the treatment goal, and the geolocation; display a filter prompt to limit the list of providers based on a criteria, wherein the criteria includes one or more of: the treatment type, the area to be treated, the treatment goal, or a distance of a provider from the list of the providers to the geolocation; receive an input in response to the filter prompt; and display a reduced list of providers based on the received input filter prompt.

### TECHNICAL FIELD

The present application relates to systems and methods for searching providers, and, in particular, to a search platform for locating providers for medical injectables.

### BACKGROUND

Searching for a qualified provider of medical injectables can be a challenging and time-consuming process. Many facilities do not prominently advertise injectable services, making it difficult to identify those offering the specific treatments needed. There is often uncertainty regarding the provider's expertise or the quality of the injectables used. Moreover, certain injectables are designed for specific purposes, for specific medications, or for particular types of anatomy, With the variety of injectable treatments available, such as neurotoxin or other specialized medications, ensuring the correct care from a trusted source becomes increasingly difficult. This lack of transparency and accessibility in finding qualified providers leads to delays in receiving timely and proper treatment.

### SUMMARY

This disclosure relates to systems and methods for locating providers for injectables.

In an aspect of the present disclosure, the system includes a search platform for providers of medical injectables, comprising a processor and a memory coupled to the processor. The memory stores one or more machine-readable instructions which, when executed by the processor, cause the system to receive a first user input indicating at least two of: a treatment type, an area to be treated, a treatment goal, or a geolocation; determine a list of providers based on the at least two of: the treatment type, the area to be treated, the treatment goal, and the geolocation; display a filter prompt to limit the list of providers based on a criteria, wherein the criteria includes at least one of: the treatment type, the area to be treated, the treatment goal, or a distance of a provider from the list of the providers to the geolocation; receive an input in response to the filter prompt; and display a reduced list of providers based on the received input filter prompt.

In an aspect of the present disclosure, the system further includes instructions that, when executed by the processor, cause the system to sort the list of providers based on at least one of: pricing, the treatment type, the area to be treated, the treatment goal, or the distance of the provider from the list of the providers to the geolocation.

In an aspect of the present disclosure, displaying the reduced list of providers includes displaying at least two of: a provider name, a pricing, the treatment type, the area to be treated, the treatment goal, or the distance of the provider from the list of the providers to the geolocation; and displaying the reduced list of providers further includes displaying an image of the area to be treated or a brand of the treatment type.

In an aspect of the present disclosure, the treatment type includes at least one of: a neurotoxin, dermal fillers, biostimulators, collagen boosters, fat dissolvers, skin boosters, thread lifts, microneedling with injectables, or lipolytics for body contouring; and the area to be treated includes at least one of: face, cheeks, neck, décolletage, hands, body, or scalp; and the treatment goal includes at least one of: wrinkle reduction, volume restoration, facial contouring, skin rejuvenation, jawline slimming, or facial balancing.

In an aspect of the present disclosure, the list of providers is further filtered based on a recommended filler for a target area; and the list of providers further includes an indication that each provider is a verified provider, and the instructions, when executed by the processor, further cause the system to provide a prompt requesting a rating for a specific provider from the list of providers, after a user selects that provider and is serviced by the provider.

In an aspect of the present disclosure, a computer-implemented method for providing search results of providers for medical injectables is included, comprising receiving a first user input indicating at least two of: a treatment type, an area to be treated, a treatment goal, or a geolocation; determining a list of providers based on the at least two of: the treatment type, the area to be treated, the treatment goal, and the geolocation; displaying a filter prompt to limit the list of providers based on a criteria, wherein the criteria includes at least one of: the treatment type, the area to be treated, the treatment goal, or a distance of a provider from the list of the providers to the geolocation; receiving an input in response to the filter prompt; and displaying a reduced list of providers based on the received input filter prompt.

In an aspect of the present disclosure, the computer-implemented method further comprises sorting the list of providers based on at least one of: pricing, the treatment type, the area to be treated, the treatment goal, or the distance of the provider from the list of the providers to the geolocation; and further comprises displaying at least two of: a provider name, a pricing, the treatment type, the area to be treated, the treatment goal, or the distance of the provider from the list of the providers to the geolocation; and further comprises displaying an image of the area to be treated or a brand of the treatment type.

In an aspect of the present disclosure, the treatment type includes at least one of: a neurotoxin, dermal fillers, biostimulators, collagen boosters, fat dissolvers, skin boosters, thread lifts, microneedling with injectables, or lipolytics for body contouring; and the area to be treated includes at least one of: face, neck, décolletage, hands, body, or scalp; and the treatment goal includes at least one of: wrinkle reduction, volume restoration, facial contouring, skin rejuvenation, jawline slimming, or facial balancing; and further comprises further filtering the list of providers based on a recommended filler for a target area.

In an aspect of the present disclosure, a non-transitory machine-readable storage medium is included, in which is stored instructions for causing a processor to execute a computer-implemented method for providing search results of providers for medical injectables, the method comprising receiving a first user input indicating at least two of: a treatment type, an area to be treated, a treatment goal, or a geolocation; determining a list of providers based on the at least two of: the treatment type, the area to be treated, the treatment goal, and the geolocation; displaying a filter prompt to limit the list of providers based on a criteria, wherein the criteria includes at least one of: the treatment type, the area to be treated, the treatment goal, or a distance of a provider from the list of the providers to the geolocation; receiving an input in response to the filter prompt; and displaying a reduced list of providers based on the received input filter prompt. These and other features will be more clearly understood from the following detailed description taken in conjunction with the accompanying drawings and claims.

Further details and aspects of exemplary aspects of the present disclosure are described in more detail below with reference to the appended figures.

### BRIEF DESCRIPTION OF THE DRAWINGS

A better understanding of the features and advantages of the disclosed technology will be obtained by reference to the following detailed description that sets forth illustrative aspects, in which the principles of the technology are utilized, and the accompanying drawings of which:

FIG. 1 is a network diagram illustration showing an exemplary system for a computer-implemented method for a search platform for providers for medical injectables, in accordance with aspects of the present disclosure;

FIG. 2 is a block diagram of a controller for the system of FIG. 1 in accordance with aspects of the present disclosure; and FIG. 3 is a flow diagram of an exemplary method for using the networked environment of FIG. 1 in accordance with aspects of the present disclosure.

### DETAILED DESCRIPTION

For purposes of promoting an understanding of the principles of the present disclosure, reference will now be made to exemplary aspects illustrated in the drawings, and specific language will be used to describe the same. It will nevertheless be understood that no limitation of the scope of the present disclosure is thereby intended. Various alterations, rearrangements, substitutions, and modifications of the inventive features illustrated herein, and any additional applications of the principles of the present disclosure as illustrated herein, which would occur to one skilled in the relevant art and having possession of this disclosure, are to be considered within the scope of the present disclosure.

In the field of cosmetic medical injectables, finding a qualified provider to administer the proper medication using the proper injectable can be a daunting and time-consuming task. The process is often complicated by the lack of transparency and accessibility in identifying providers who offer specific injectable treatments. Many facilities do not prominently advertise their injectable services, making the task difficult for patients to locate providers who meet their specific needs. Additionally, there is often uncertainty regarding the expertise of the provider or the quality or type of the injectables used, which can lead to delays in receiving timely and appropriate treatment.

Existing solutions in the market have several disadvantages. Traditional methods of finding providers, such as word-of-mouth recommendations or basic online searches, do not offer comprehensive information about the provider's qualifications, experience, or the specific treatments the provider offers. These methods often lack the ability to filter providers based on specific criteria such as treatment type and relevant injectable, area to be treated and relevant injectable, or the provider's proximity to the patient. Furthermore, current platforms do not effectively integrate advanced technologies to enhance the search and booking process, resulting in inefficiencies and a lack of personalized recommendations for patients.

The present disclosure solves these technical problems by introducing a novel system and method for a search platform specifically designed for providers of medical injectables. This approach leverages advanced technologies to create a seamless, efficient, and trustworthy system for patients seeking injectable treatments. The platform enables patients to input specific criteria, such as treatment type, area to be treated, and geolocation, to generate a tailored list of qualified providers who are authorized to use the correct injectable based on the queried criteria. By incorporating features such as AI-driven recommendations, real-time booking, and verified provider reviews, the system significantly enhances the patient experience, ensuring that patients can easily find and book appointments with trusted providers who meet their specific needs.

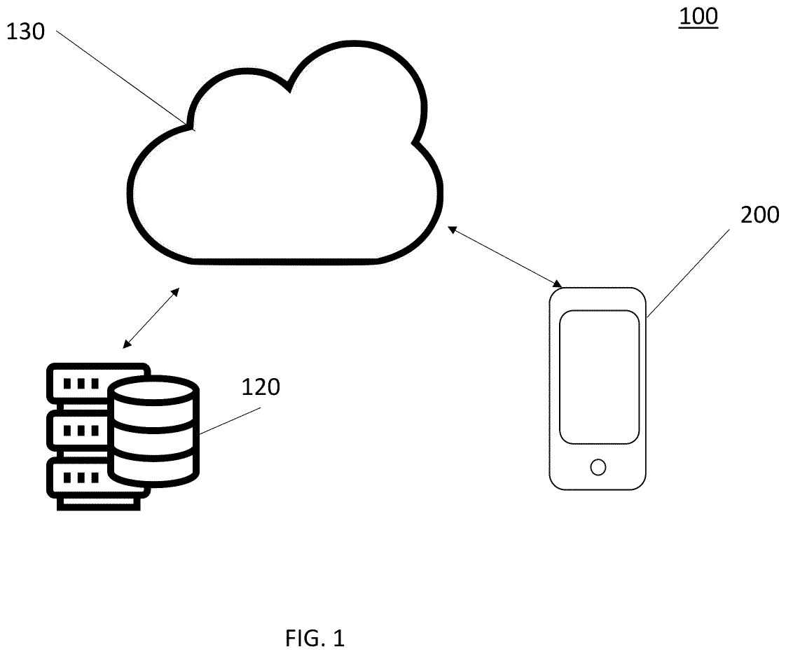

Referring to FIG. 1, an exemplary system 100 in accordance with aspects of the present disclosure includes a patient device 200. It is contemplated that the patient device 200 may run programs locally on the patient device 200, remotely via a network 130, over the cloud, and/or on a remote server 120. The patient device 200 may include, but is not limited to, a mobile phone, a personal computer, a tablet, and/or other handheld device.

The network 130 may be wired or wireless and can utilize technologies such as Wi-Fi®, Ethernet, Internet Protocol, 3G, 4G, 5G, TDMA, CDMA, or other communication technologies. The network 150 may include, for example, but is not limited to, a cellular network, residential broadband, satellite communications, private network, the Internet, local area network, wide area network, storage area network, campus area network, personal area network, or metropolitan area network.

The term “application” may include a computer program and/or machine-readable instructions designed to perform particular functions, tasks, or activities for the benefit of a patient. Application may refer to, for example, software running locally or remotely, as a standalone program or in a web browser, or other software that would be understood by one skilled in the art to be an application. An application may run on the patient device 200, a server, or on a patient device, including, for example, on a mobile device 140 or a patient computer system 110. The configuration of FIG. 1 is exemplary, and variations are contemplated to be within the scope of the present disclosure.

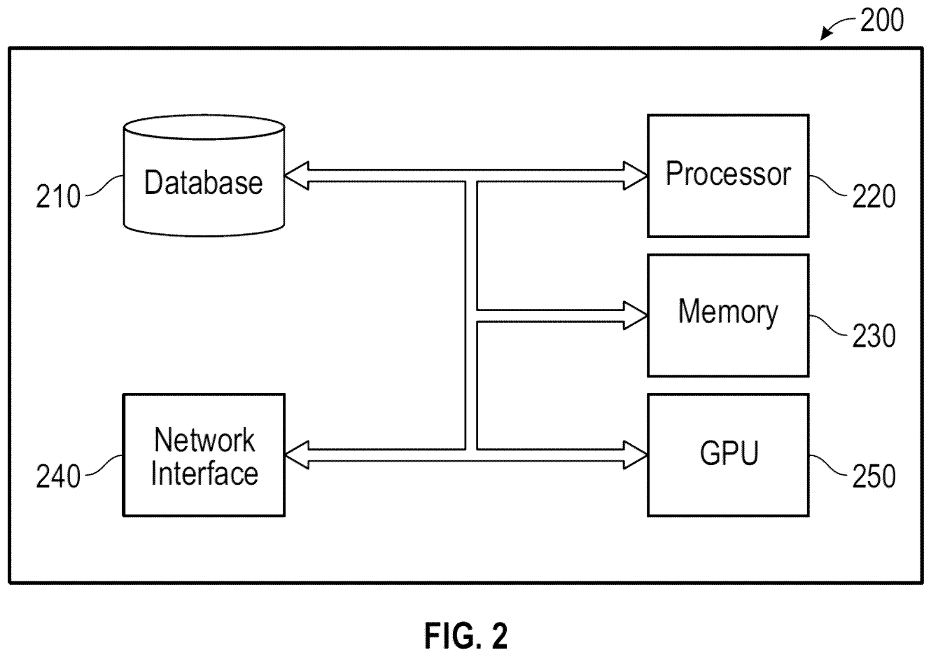

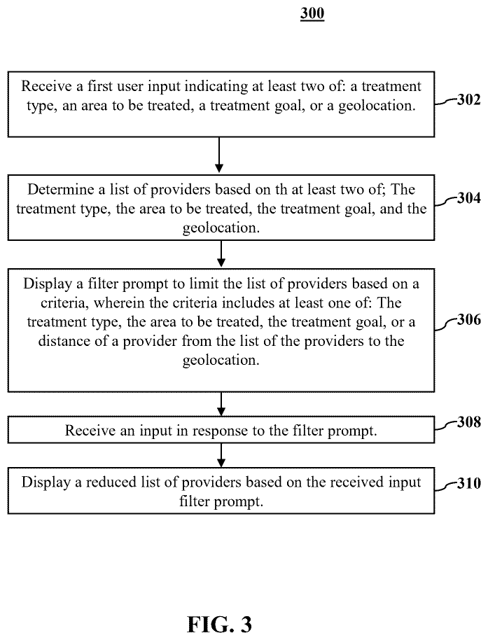

Referring now to FIG. 2, exemplary components in the patient device 200 in accordance with aspects of the present disclosure include, for example, a database 210, one or more processors 220, at least one memory 230, and a network interface 240. In aspects, the patient device 200 may include a graphical processing unit (GPU) 250, which may be used for processing machine learning models.

The database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently hold digital data until purposely erased, such as flash memory, magnetic devices such as hard disk drives, and optical media such as a CD, DVD, Blu-ray disc, or the like.

In various aspects, data may be stored on the patient device 200, including, for example, patient preferences, historical data, and/or other data. The data can be stored in the database 210 and sent via the system bus to the processor 220.

As will be described in more detail later herein, the processor 220 executes various processes based on instructions that can be stored in the server memory 230 and utilizing the data from the database 210. With reference also to FIG. 1, a request from a patient device, such as a mobile device or a patient computer, can be communicated to the server through the server's network interface 240. The illustration of FIG. 2 is exemplary, and persons skilled in the art will be understood other components that may exist in a patient device 200. Such other components are not illustrated in FIG. 2 for clarity of illustration.

FIG. 3 shows a block diagram for an exemplary method for search platform for locating providers for injectables. Although the steps of FIG. 3 are shown in a particular order, the steps need not all be performed in the specified order, and certain steps can be performed in another order. For example, FIG. 3 will be described below with a server (not shown) performing the operations. However, in various aspects, the operations of FIG. 3 may be performed all or in part by the patient device 200 of FIG. 1. In aspects, the operations of FIG. 3 may be performed all or in part by another device, for example, a mobile device and/or a patient computer system. These and other variations are contemplated to be within the scope of the present disclosure. In aspects, the injectables may be medical and/or cosmetic injectables.

Initially, at step 302, the patient device 200 receives a first user (patient) input indicating at least two of: a treatment type, an area to be treated, a treatment goal, or a geolocation. For example, the geolocation may be taken from the user device's GPS location or may be entered, for example, as a zip code or as an indication on a map.

In aspects, the treatment type may include, for example, a neurotoxin, dermal fillers, biostimulators, collagen boosters, fat dissolvers, skin boosters, thread lifts, microneedling with injectables, and/or lipolytics for body contouring

At step 304, the user device 200 determines a list of providers based on the at least two of: the treatment type, the area to be treated, the treatment goal, and the geolocation.

In aspects, the user device 200 may sort the list of providers based on at least one of: pricing, the treatment type, the area to be treated, the treatment goal, or the distance of the provider from the list of the providers to the geolocation.

At step 306, the user device 200 displays a filter prompt to limit the list of providers based on a criteria, wherein the criteria includes at least one of: the treatment type, the area to be treated, the treatment goal, or a distance of a provider from the list of the providers to the geolocation.

The system 100 enables users to filter providers based on their specialization in single or combination treatments. Patients (users) can refine searches to locate providers who exclusively offer neurotoxin treatments, dermal fillers, or a combination of both. Additional filtering options include providers specializing in biostimulators and collagen boosters such as Sculptra®, Radiesse®, and PRP, as well as those offering a comprehensive anti-aging approach that integrates neurotoxins, fillers, threads, biostimulators, and skin boosters.

Patients (users) may filter injectors (e.g., providers) based on the best filler for their target area. For example, the target area (i.e., area to be treated) may include face, cheeks, neck, décolletage, hands, body, or scalp.

A variety of dermal fillers are recommended for different facial and body areas to enhance volume, contouring, and skin smoothing. For lip augmentation, suggested fillers include RHA 2®, RHA 3®, Restylane Kysse®, Juvederm Ultra XC®, Juvederm Volbella®, Revanesse Versa®, and Revanesse Lips+®. In the cheek area, volumizing fillers such as Juvederm Voluma®, Restylane Lyft®, Restylane Contour®, RHA 4®, Radiesse®, and Sculptra® provide structural support and restoration.

For the correction of nasolabial folds, also known as smile lines, recommended fillers include Juvederm Vollure®, Juvederm Ultra Plus®, Restylane-L®, Restylane Refyne®, Restylane Defyne®, RHA 3®, and Revanesse Versa®. Marionette lines, which extend from the mouth downward, can be treated with Juvederm Ultra Plus®, Juvederm Vollure®, Restylane Defyne®, and RHA 3®. Under-eye rejuvenation, specifically targeting tear troughs, can benefit from Restylane Eyelight, Juvederm Volbella®, RHA Redensity®, and Belotero Balance®.

For jawline and chin augmentation, suitable fillers include Juvederm Voluma®, Restylane Lyft®, RHA 4®, Radiesse®, and Sculptra®. Temple volume restoration is effectively addressed with Juvederm Voluma®, Restylane Lyft®, Radiesse®, and Sculptra®. Hand rejuvenation to restore lost volume can be performed using Radiesse® and Restylane Lyft®. Fine line smoothing and skin boosting treatments may incorporate Juvederm Skinvive®, RHA Redensity®, Belotero Balance®, and Radiesse® Hyperdilute. Body contouring applications involve the use of Sculptra® for buttocks and hip dips and Radiesse® in a hyperdilute form for skin tightening.

Neurotoxin treatments are categorized into on-label and off-label applications. On-label treatments include glabellar lines (frown lines), forehead lines, and crow's feet. Off-label uses extend to brow lifts, bunny lines, lip flips, gummy smiles, downturned mouth corners (DAO), pebbled chin (mentalis muscle), jawline slimming and masseter reduction, platysmal bands (neck lift), Nefertiti lift for jawline contouring, hyperhidrosis management for hands, feet, and underarms, trapezius slimming (Trap Tox), calf slimming, shoulder and neck pain reduction, migraine and TMJ relief, smoker's lines (lipstick lines), under-eye jelly rolls, and nasal tip lifts.

To refine search preferences, patients may filter results based on provider credentials such as MD, RN, NP, PA, or DDS, as well as years of experience, star ratings and reviews, promotional offers, combination treatments, before-and-after galleries, distance and location, and virtual consultation availability.

At step 308, the patient device 200 receives an input in response to the filter prompt.

At step 310, the patient device 200 displays a reduced list of providers based on the received input filter prompt. In aspects, the reduced list of providers may include: a provider name, a pricing, the treatment type, the area to be treated, the treatment goal, and/or the distance of the provider from the list of the providers to the geolocation.

In aspects, displaying the reduced list of providers further includes displaying an image of the area to be treated and/or displaying a brand of the treatment type.

Certain treatment areas benefit from multiple modalities, depending on the patient's aesthetic concerns and desired outcomes. The search engine categorizes provider based on their expertise in administering neurotoxins, fillers, or both in combination, allowing for a targeted and efficient provider selection process. Providers who offer both Botox® and fillers for the same treatment area are labeled as “Combination Treatment Experts,” while those incorporating collagen stimulators alongside neurotoxins or fillers receive the designation of “Full-Face Anti-Aging Experts.” Search filters further classify providers into categories such as “Botox Only Providers,” “Filler Only Providers,” “Botox +Filler Combination Providers,” and “Collagen Stimulator+Botox or Filler.”

A comparison feature allows patients to evaluate two providers side by side, analyzing treatment experience, star ratings, reviews, online booking availability, complimentary consultations, pricing, and other relevant factors. The system integrates advanced search functionalities, including filtering by provider type, treatment area, and product usage. Patients can also refine results based on provider experience, reviews, promotions, before-and-after galleries, and virtual consultation availability. Providers may also include, but are not limited to, for example, plastic surgery and esthetic providers (estheticians can perform chemical peels, lasers, IPL, skin rejuvenation, clear and brilliant, moxi, fraxel).

To further enhance the search engine, the system 100 may include AI-driven provider matching, enabling personalized recommendations based on uploaded facial images or questionnaire responses. The AI system suggests top-rated providers specializing in the desired treatments, recommends combination therapies, and provides before-and-after comparisons for similar cases. This feature streamlines the selection process, particularly for first-time patients unsure of their treatment needs.

For example, the patient may initially upload a facial image or complete a detailed questionnaire. The questionnaire might include questions about the patient's aesthetic goals, areas of concern, previous treatments, and any specific preferences or constraints (e.g., budget, location). If a facial image is uploaded, the system may use computer vision and machine learning algorithms to analyze the facial structure, identifying key features such as skin texture, volume loss, and areas that could benefit from enhancement or correction. The system processes the data from the image analysis or questionnaire responses to understand the patient's unique needs and preferences. This might involve assessing the symmetry of facial features, identifying potential treatment areas, and understanding the patient's desired outcomes. The AI-driven recommendation engine uses the processed data to match the patient with suitable providers. This involves comparing the patient's profile with a database of providers, considering factors such as the provider's expertise, experience with similar cases, and availability of specific treatments. The system 100 may generate a list of recommended providers, for example, prioritizing those who have a proven track record with the patient's specific needs. The recommendations may include details about the provider's qualifications, patient reviews, treatment options, and pricing. The patient can review the recommendations and provide feedback, which the system 100 can use to refine future suggestions. This iterative process helps improve the accuracy and relevance of the recommendations over time.

By incorporating AI-driven provider matching, the system 100 offers a highly personalized and efficient way for patients to find the most suitable providers for their injectable treatments, enhancing patient satisfaction and treatment outcomes.

The system 100 may include a real-time availability and instant booking feature configured to allow patients to view provider schedules and book appointments instantly. Filters may include, for example, immediate openings, weekend versus weekday availability, and virtual consultation options prior to in-person visits. This feature minimizes the need for direct communication with multiple offices and assists providers in optimizing their schedules by filling last-minute cancellations.

A subscription-based VIP membership program is also offered, providing patients with exclusive benefits such as discounted treatments, priority booking, early access to promotions, birthday discounts on Botox® or fillers, and loyalty rewards redeemable for future treatments. This program fosters customer retention and engagement while benefiting providers by encouraging long-term patient relationships.

Additionally, the search engine includes a directory of aesthetic provider training and certification programs, further supporting industry professionals by providing resources for continuing education and accreditation. These enhancements collectively establish a highly effective, patient-friendly search engine tailored to the needs of both providers and patients seeking aesthetic treatment.

A specialized section is provided for providers to list certifications and advanced training courses, access available training workshops or masterclasses from top trainers, and rate and review industry training programs. This allows new providers to identify the best trainers while enabling patients to assess the qualifications of highly trained providers.

An AI-powered virtual treatment preview utilizing augmented reality (AR) enables patients to visualize potential treatment results on their own faces. By uploading a photo, patients can toggle between different treatment options, such as varying amounts of lip filler, and instantly compare before-and-after results. This technology assists first-time patients in visualizing outcomes and increases confidence in booking treatments.

A treatment cost estimator powered by AI allows patients to input desired treatments and budget constraints. The system 100 estimates pricing based on providers in the patient's area, suggests promotions or bundled deals for savings, and offers financing options if available through providers. This functionality helps patients budget for treatments in advance and reduces hesitation related to pricing concerns.

A side-by-side provider comparison tool provides patients with the ability to compare providers based on pricing, experience, certifications, before-and-after results, average ratings, number of reviews, techniques offered (e.g., needle vs. cannula, microdosing), and types of neurotoxins and fillers used. This consolidated data assists patients in making informed decisions efficiently.

An exclusive patient review system ensures that only individuals who have received treatments through the system 100 can submit reviews. Verified before-and-after images can be tagged to specific provider, and top-rated provider receive a “Verified Provider” badge. This system enhances trust and credibility while eliminating fake reviews.

A business management dashboard allows providers to manage appointments and availability, track revenue and patient bookings, offer exclusive promotions to VIP members, and list training courses they provide. This system 100 enhances business efficiency for providers while streamlining interactions with patients.

A dedicated mobile application with push notifications provides appointment reminders, discount alerts for promotions offered by selected providers, and maintenance reminders for treatments such as Botox touch-ups or filler maintenance. This feature improves patient engagement and encourages repeat bookings through seamless scheduling and notifications.

An advanced filtering system allows patients to search for providers based on specialty areas, including lip filler specialists, jawline contouring experts, full-facial balancing providers, preventative Botox specialists, male aesthetic providers, and experts in ethnic beauty adaptations tailored to different skin types and facial structures. This ensures patients find specialists suited to their specific needs, leading to higher satisfaction and improved treatment outcomes.

The introduction of AI-driven recommendations, real-time booking, a patient membership model, and provider business tools differentiates this search engine within the aesthetic industry. Unique features include combination treatment filters (e.g., Botox and filler), AI-powered provider matching and cost estimation, an AR-based “try before you buy” visualization tool, real-time appointment booking with instant promotions, a mobile engagement app with push notifications, and a verified review system with provider ratings.

The proposed technology addresses inefficiencies in the aesthetic and cosmetic injection industry by providing a structured, AI-enhanced solution. Existing platforms lack advanced filtering capabilities, combination treatment matching, and real-time appointment booking, leading to inefficiencies in provider searches, fragmented pricing visibility, and a lack of transparency in treatment options. The proposed system introduces precision filtering for treatment categories, provider expertise, provider techniques, and real-time availability.

An AI-powered recommendation engine analyzes patient-uploaded images or questionnaire responses to assess facial structure, aesthetic goals, and budget, generating personalized treatment recommendations and provider matches. The AI-powered recommendation engine may use a convolutional neural network to assess facial structure, aesthetic goals, and budget, generating personalized treatment recommendations and provider matches. Combination treatment recognition allows patients to identify providers specializing in multiple treatments for the same area, streamlining provider selection and consultation efficiency. Real-time booking functionality integrates provider availability, reducing manual scheduling inefficiencies and last-minute cancellations. Augmented reality visualization offers patients a preview of potential treatment results, increasing confidence in treatment selection. Transparent pricing aggregation consolidates provider rates, package deals, and promotional offers, enabling patients to make cost-effective choices.

A verified patient review system ensures authenticity by allowing only patients who have booked through the system 100 to leave reviews. Providers' before-and-after images are linked to actual treatments performed through the system, increasing transparency and credibility.

This technology-driven solution directly addresses industry inefficiencies by enhancing precision in provider searches, integrating AI for predictive treatment matching, eliminating friction in booking processes, and ensuring trust and transparency in provider reviews. The combination of AI-powered search capabilities, real-time appointment booking, verified patient reviews, and transparent pricing structures establishes a comprehensive and innovative solution within the aesthetic industry.

A specialized search engine has been developed to address inefficiencies in the aesthetic industry by optimizing patient-provider matching and integrating educational tools. This technology allows for the identification of providers specializing in multi-modality treatments, ensuring that patients seeking combination procedures such as Botox and filler can locate providers capable of performing both treatments in a single visit. By eliminating the need for multiple consultations, patient convenience is enhanced while providers benefit from attracting a more targeted clientele. Additionally, the system reduces the time spent on phone inquiries by clearly listing provider information, including services, pricing, and product availability. This enables providers to focus on performing treatments rather than handling unnecessary calls.

This technology significantly improves the patient experience by minimizing the frustration associated with booking providers who do not align with specific treatment needs. Patients save time by identifying suitable providers, gain confidence in their treatment choices, and can book appointments without back-and-forth communication. For providers, efficiency increases as last-minute cancellations due to product mismatches are reduced, and non-relevant inquiries are minimized. The ability to attract patients actively seeking specific expertise results in improved patient satisfaction and optimized appointment scheduling.

A major challenge in the aesthetic industry is the prevalence of misinformation and a lack of patient education, leading to booking errors and treatment hesitation. Many patients incorrectly assume that Botox® can address volume loss or deep-set wrinkles, leading to misguided bookings that require re-education during consultations. Additionally, misinformation from non-medical sources often results in patients requesting treatments unsuitable for their concerns, further complicating the consultation process. These misconceptions contribute to wasted appointment slots, patient frustration, and lost revenue for providers.

To resolve these inefficiencies, the system may include an AI-powered educational system. The AI-powered educational system may use a large language model. Patients may complete a guided questionnaire that assesses their aesthetic goals, and the system provides recommendations based on the most suitable treatments. If an inappropriate treatment is selected, an explanation is provided before booking, ensuring that patients are informed before consultations. A side-by-side treatment comparison feature further clarifies differences in expected results, longevity, and downtime, reducing the likelihood of cancellations. Interactive educational content, including videos and articles, explains treatment methodologies, facial anatomy, and optimal sequencing to improve patient understanding before booking. An AI-powered virtual assistant addresses common patient questions, minimizing the volume of phone inquiries for providers.

Filtering capabilities ensure that patients searching for treatments such as Botox for jowls receive guidance on alternative procedures, such as filler, when appropriate. The system automatically lists providers offering both treatments, guaranteeing that patients book with professionals capable of delivering the best results. By addressing the root causes of booking errors and misinformation, this technology increases patient confidence, reduces hesitation, and enhances overall efficiency.

Expanding beyond injectable treatments, the search engine now incorporates a comprehensive breakdown for esthetician services, enabling patients to locate providers specializing in non-invasive treatments. Advanced filtering options allow patients to search for licensed estheticians, medical estheticians, and higher-licensed providers working under physicians. This refined categorization enhances accessibility, ensuring that patients receive services from qualified professionals while improving business operations for providers. Through AI-driven recommendations, treatment filtering, and integrated educational tools, this system establishes a new standard in the aesthetic industry by improving transparency, reducing inefficiencies, and optimizing patient decision-making.

The disclosed system enables, for example, esthetician services by incorporating a comprehensive filtering system that allows patients to identify providers offering non-invasive treatments. The system 100 facilitates searches based on treatment type, provider specialization, licensing level, treatment area, provider qualifications, experience, pricing, and promotional offers. The treatment-based search categorizes services into facial treatments, peels and exfoliation, microneedling, collagen stimulation, skin tightening, laser treatments, and body treatments, enabling precise filtering based on individual skincare needs. Additionally, the system 100 enables filtering based on provider licensing, distinguishing between licensed estheticians, medical estheticians, and those operating under physician supervision.

An advanced search function categorizes providers based on areas of treatment, allowing patients to locate services tailored to specific facial or body regions. The system further refines search results by provider qualifications, including licensing level, years of experience, treatment volume, before-and-after galleries, and certifications in specialized procedures. Pricing transparency is achieved through filters for cost ranges, package deals, seasonal promotions, and loyalty discounts. Addressing common inefficiencies in the industry, the system integrates an AI-driven recommendation engine that analyzes patient input to suggest the most appropriate treatments.

A tagging system ensures clarity in provider specialization and licensing, reducing ambiguity regarding service availability. A side-by-side comparison tool enables patients to evaluate multiple treatment options based on efficacy, cost, and provider reviews. Real-time booking integration streamlines scheduling, eliminating the need for direct inquiries regarding availability and pricing. The system further incorporates state-specific licensing regulations, ensuring compliance by restricting treatment options based on jurisdictional limitations.

The system 100 is also configured to support searches related to plastic surgery services, allowing patients to locate, compare, and book procedures based on surgical expertise, board certification, pricing, combination surgeries, and real-time appointment availability. The filtering mechanism classifies procedures into facial, breast, and body surgeries, as well as non-surgical and minimally invasive options. Patients can refine searches based on board certification, specialization, years of experience, case volume, and surgeon-provided before-and-after images. A dedicated function facilitates searches for combination surgeries, optimizing patient outcomes by allowing multiple procedures to be performed within a single operation.

A transparent pricing structure categorizes providers into different cost tiers and integrates financing options such as in-house payment plans and third-party financing solutions. An AI-powered recommendation system further enhances usability by analyzing patient needs and suggesting suitable surgical procedures. The integration of these features ensures efficiency in provider matching, treatment comparison, and scheduling, improving accessibility and decision-making for patients seeking esthetician and plastic surgery services.

In aspects, system 100 may provide an advanced search and booking platform for plastic surgery procedures, integrating AI-driven personalized matching, real-time availability, and transparent provider information. The system enables patients to complete a facial or body assessment, after which AI analyzes the input to recommend the most suitable surgical options aligned with individual aesthetic goals. Based on the analysis, the system determines whether a surgical or non-surgical approach, such as fillers or skin tightening treatments, would be more appropriate.

To enhance patient confidence before booking, an AI-powered before-and-after visualization tool projects expected surgical results by analyzing real patient outcome data. The system categorizes surgical procedures based on expected recovery time, allowing patients to filter options that fit their lifestyle. Procedures with minimal downtime, including eyelid surgery, mini facelift, chin implant, lip lift, and fat transfer to the face, are distinguished from those requiring moderate downtime, such as rhinoplasty, breast augmentation, and liposuction. Extended recovery procedures, including facelift with neck lift, mommy makeover, Brazilian butt lift, and tummy tuck, are also categorized for efficient filtering.

A real-time booking system streamlines appointment scheduling by displaying surgeon availability, eliminating the need for phone inquiries. The system also incorporates last-minute openings and cancellation notifications, allowing patients to secure consultations at the earliest opportunity. Verified patient reviews and before-and-after galleries enhance trust in provider selection, ensuring that only authenticated patients can leave feedback and that surgical results are linked directly to the respective provider's profile.

A comparison tool enables side-by-side evaluation of surgeons based on experience level, before-and-after results, pricing, and recovery times. The system further enhances the search process through surgical specialty filtering, allowing patients to refine searches based on specific areas such as facial, breast, body, or revision surgeries. Additionally, real-time pricing transparency eliminates hidden fees, while financing options, including in-house payment plans and third-party financing solutions, are integrated for accessibility.

The system supports combination surgery searches, matching patients with surgeons who specialize in multiple procedures, including mommy makeovers, facelift and neck lift combinations, tummy tuck with liposuction, and breast augmentation with lift. AI-driven surgical recommendations further enhance decision-making by comparing options such as facelift versus thread lift or tummy tuck versus non-invasive skin tightening based on individual patient goals.

Search functionality includes filtering by surgeon specialization, experience level, and procedure volume, ensuring patients connect with highly experienced providers. Options include filtering based on the number of surgeries performed, years of experience, and verified before-and-after case studies. A dedicated revision surgery search feature identifies surgeons specializing in corrective procedures, such as revision rhinoplasty or facelift correction.

Pricing filters allow patients to search based on estimated surgery costs, available payment plans, and insurance eligibility for medically necessary procedures. The system's 100 AI-assisted visualization further enhances patient confidence by generating personalized previews of potential surgical outcomes based on historical patient data.

The disclosed system represents a significant advancement in the plastic surgery industry by eliminating consultation mismatches, increasing transparency in surgeon experience and pricing, and optimizing efficiency for both patients and providers. The integration of AI-driven matching, before-and-after visualization, real-time scheduling, and verified patient reviews ensures an informed decision-making process, improving patient outcomes while streamlining business operations for plastic surgeons.

The technical problem addressed by the search platform for providers of medical injectables is the inefficiency and lack of transparency in locating qualified providers for specific injectable treatments. Traditional methods, such as word-of-mouth recommendations or basic online searches, do not provide comprehensive information about a provider's qualifications, experience, or the specific treatments they offer. This results in a time-consuming and often frustrating process for patients, who may face uncertainty regarding the expertise of the provider or the quality of the injectables used. Additionally, existing platforms lack advanced filtering capabilities and do not effectively integrate technologies to enhance the search and booking process, leading to delays in receiving timely and appropriate treatment. The system 100 solves these issues by offering a patient centric, AI-driven solution that streamlines the process of finding, comparing, and booking appointments with trusted providers.

The system 100 provides the practical application of an implementation as a digital tool that enhances the process of connecting patients with qualified medical professionals for injectable treatments. This system 100 can be used by patients to efficiently locate and book appointments with providers who meet specific criteria, such as treatment type, location, and expertise, thereby improving the overall patient experience and ensuring timely access to necessary medical services. By leveraging AI-driven recommendations and real-time booking capabilities, the platform provides a tangible technological solution to the problem of finding and selecting appropriate medical providers, demonstrating a concrete and useful application of the underlying technology.

In aspects, system 100 may include an educational tool configured to teach patients which treatment they would need to obtain a desired result. For example, often, patients come in asking for Botox® on their lower face (a 5-minute treatment with no healing time) to treat their jowls. When they require a dermatology filler (i.e., a 30-minute treatment with bruising and swelling). The patient may end up either not getting the treatment or must come back another time because they have an event or plans within the week. This is just one example of why it would be important. The educational tool may use a large language model (LLM) to query the patient on their desired result and provide assistance by recommending to the patient which treatment they would need to obtain a desired result. For example, the patient may indicate to the system that they would like to treat their jowls. The system 100 may feed the query into the LLM, which may map the query into a semantic space. The LLM may access additional data such as scientific articles and/or websites to use as context. The LLM may then indicate to the patient the appropriate treatment to get the indicated desired result. Although an LLM is used as an example, other algorithms may be used.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages that are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above are also intended to be within the scope of the disclosure.

1. A system for a search platform for providers for medical injectables, comprising:
a processor; and
a memory coupled to the processor and storing one or more machine-readable instructions which, when executed by the processor, cause the system to:
receive a first user input indicating at least two of: a treatment type, an area to be treated, a treatment goal, or a geolocation;
receive patient-specific information including at least one of a patient-uploaded image or questionnaire responses;
process the patient-specific information using a machine learning model to generate a patient profile including at least one of a patient-specific treatment area or a patient-specific desired outcome;
determine a list of providers based on the at least two of: the treatment type, the area to be treated, the treatment goal, and the geolocation, wherein determining the list of providers includes comparing the patient profile with provider records in a provider database based on at least one of provider expertise, experience with similar cases, or availability of specific treatments;
display a filter prompt to limit the list of providers based on a criteria, wherein the criteria includes at least one of: the treatment type, the area to be treated, the treatment goal, or a distance of a provider from the list of the providers to the geolocation;
receive an input in response to the filter prompt; and
display a reduced list of providers based on the received input in response to the filter prompt.

2. The system of claim 1, wherein the instructions, when executed by the processor, further cause the system to:
sort the list of providers based on at least one of: pricing, the treatment type, the area to be treated, the treatment goal, or the distance of the provider from the list of the providers to the geolocation.

3. The system of claim 1, wherein displaying the reduced list of providers includes displaying at least two of: a provider name, a pricing, the treatment type, the area to be treated, the treatment goal, or the distance of the provider from the list of the providers to the geolocation.

4. The system of claim 3, wherein displaying the reduced list of providers further includes displaying an image of the area to be treated.

5. The system of claim 3, wherein displaying the reduced list of providers further includes displaying a brand of the treatment type.

6. The system of claim 1, wherein the treatment type includes at least one of: a neurotoxin, dermal fillers, biostimulators, collagen boosters, fat dissolvers, skin boosters, thread lifts, microneedling with injectables, or lipolytics for body contouring.

7. The system of claim 1, wherein the area to be treated includes at least one of: face, cheeks, neck, décolletage, hands, body, or scalp.

8. The system of claim 1, wherein the treatment goal includes at least one of: wrinkle reduction, volume restoration, facial contouring, skin rejuvenation, jawline slimming, or facial balancing.

9. The system of claim 1, wherein the list of providers is further filtered based on a recommended filler for a target area.

10. The system of claim 1, wherein the list of providers further includes an indication that each provider is a verified provider, and
wherein the instructions, when executed by the processor, further cause the system to:
provide a prompt requesting a rating for a specific provider from the list of providers, after a user selects that provider and is serviced by the provider.

11. A computer-implemented method for providing search results of providers for medical injectables, the method comprising:
receiving a first user input indicating at least two of: a treatment type, an area to be treated, a treatment goal, or a geolocation;
receiving patient-specific information including at least one of a patient-uploaded image or questionnaire responses;
processing the patient-specific information using a machine learning model to generate a patient profile including at least one of a patient-specific treatment area or a patient-specific desired outcome;
determining a list of providers based on the at least two of: the treatment type, the area to be treated, the treatment goal, and the geolocation, wherein determining the list of providers includes comparing the patient profile with provider records in a provider database based on at least one of provider expertise, experience with similar cases, or availability of specific treatments;
displaying a filter prompt to limit the list of providers based on a criteria, wherein the criteria includes at least one of: the treatment type, the area to be treated, the treatment goal, or a distance of a provider from the list of the providers to the geolocation;
receiving an input in response to the filter prompt; and
displaying a reduced list of providers based on the received input in response to the filter prompt.

12. The computer-implemented method of claim 11, further comprising:
sorting the list of providers based on at least one of: pricing, the treatment type, the area to be treated, the treatment goal, or the distance of the provider from the list of the providers to the geolocation.

13. The computer-implemented method of claim 11, further comprising:
displaying at least two of: a provider name, a pricing, the treatment type, the area to be treated, the treatment goal, or the distance of the provider from the list of the providers to the geolocation.

14. The computer-implemented method of claim 13, further comprising:
displaying an image of the area to be treated.

15. The computer-implemented method of claim 13, further comprising:
displaying a brand of the treatment type.

16. The computer-implemented method of claim 11, wherein the treatment type includes at least one of: a neurotoxin, dermal fillers, biostimulators, collagen boosters, fat dissolvers, skin boosters, thread lifts, microneedling with injectables, or lipolytics for body contouring.

17. The computer-implemented method of claim 11, wherein the area to be treated includes at least one of: face, neck, décolletage, hands, body, or scalp.

18. The computer-implemented method of claim 11, wherein the treatment goal includes at least one of: wrinkle reduction, volume restoration, facial contouring, skin rejuvenation, jawline slimming, or facial balancing.

19. The computer-implemented method of claim 11, further comprising:
further filtering the list of providers based on a recommended filler for a target area.

20. A non-transitory machine-readable storage medium in which is stored instructions for causing a processor to execute a computer-implemented method for providing search results of providers for medical injectables, the method comprising:
receiving a first user input indicating at least two of: a treatment type, an area to be treated, a treatment goal, or a geolocation;
receiving patient-specific information including at least one of a patient-uploaded image or questionnaire responses;
processing the patient-specific information using a machine learning model to generate a patient profile including at least one of a patient-specific treatment area or a patient-specific desired outcome;
determining a list of providers based on the at least two of: the treatment type, the area to be treated, the treatment goal, and the geolocation, wherein determining the list of providers includes comparing the patient profile with provider records in a provider database based on at least one of provider expertise, experience with similar cases, or availability of specific treatments;
displaying a filter prompt to limit the list of providers based on a criteria, wherein the criteria includes at least one of: the treatment type, the area to be treated, the treatment goal, or a distance of a provider from the list of the providers to the geolocation;
receiving an input in response to the filter prompt; and
displaying a reduced list of providers based on the received input in response to the filter prompt.
